(19) United States
(12) Patent Application Publication
(10) Pub. No.: US 2021/0137718 A1
(43) Pub. Date: May 13, 2021

(54) HOLD-UP DEVICE FOR SECURING LEG OR KNEE BRACES / IMMOBILIZERS

(71) Applicant: William W Faloon, Spokane, WA (US)

(72) Inventor: William W Faloon, Spokane, WA (US)

(21) Appl. No.: 17/089,700

(22) Filed: Nov. 4, 2020

Related U.S. Application Data
(60) Provisional application No. 62/930,531, filed on Nov. 4, 2019.

(51) Int. Cl. A61F 5/01

(52) U.S. Cl. CPC A61F 5/0109; A61F 5/0125

(57) ABSTRACT

Hold-up devices for securing a leg brace from sliding or rotating on a patient's leg are provided. In various embodiments, a strap system for securing a leg brace includes at least one strap member for securing the hold-up device to a belt or waistband and includes multiple selectable mechanisms for attachment to a leg brace. In an example use scenario, a hold-up device has an adjustable strap, straps or strap-like member attachable to a belt, waistband or belt-like member around the patient's waist. The example hold-up device is connectable in various ways to the leg brace to prevent the brace from sliding down the patient's leg, from rotating on the patient's leg, or from both sliding and rotating on the patient's leg.

### RELATED APPLICATIONS

This patent application claims the benefit of priority to U.S. Provisional Patent Application No. 62/930,531 to Faloon, filed Nov. 4, 2019 and incorporated herein by reference in its entirety.

### BACKGROUND

Knee immobilizers are orthotic braces or splints that commonly wrap around part of a user's leg, secured to itself by straps with hook and loop fastener material, zippers, buckles, or other self-fastening mechanisms. Such leg braces, immobilizers and orthopedic appliances for the lower limb come in many variations and assume many names besides “knee immobilizer,” such as leg stabilizer, full leg brace, orthopedic leg brace, leg splint, immobilizer brace, patellofemoral support brace, knee range of motion braces and many other identifiers. The term “braces” is used loosely herein to refer collectively and representatively to these various devices.

Braces are used after injuries, surgery or other conditions that require support for the knee or leg, including during physical therapy. They have the disadvantage of having some weight and a propensity to slide down the leg since, in many cases, the human leg tapers narrower from the hip to the foot. Some braces can also rotate inadvertently and cause chafing of the skin at some points of contact. The tendency for braces to slide down the leg is sometimes countered with a shoulder strap that functions as a suspender. This can be awkward because it spans from the shoulder to below the waist which is ineffective if there is bending at the user's waist and it does not address rotation of the braces. Shoulder strapping is also not very adjustable.

### SUMMARY

Hold-up devices for securing a leg brace from sliding or rotating on a patient's leg are provided. In various embodiments, a strap or strap system for securing a leg brace includes at least one strap member for securing the hold-up device to a belt or waistband and includes multiple selectable mechanisms for attachment to a leg brace. An example hold-up device has an adjustable strap, straps, or strap-like member attachable to a belt, waistband or belt-like member that is around a patient's waist. The example hold-up device is connectable in various ways to the leg brace to keep it from sliding down or rotating on the patient's leg. The multiple attachment mechanisms may include a choice of one or more carabiner clips on the strap-like member for connecting to a loop or strap on the leg brace and a clip or clasp on the strap-like member for grasping material of the given leg brace. The hold-up device may have a buckle or an elastic material to adjust its length or tension.

In an implementation, the hold-up device may have an auxiliary piece to base a lifting force of the hold-up device from a point of the hip joint or planes of the hip joint on the corresponding side that a patient would be wearing the leg brace. In an implementation, locating the anchoring point for securing the leg brace in transecting planes of the actual hinging and pivoting motion of the corresponding human hip joint allows one or more securing straps to remain taut at a fixed length over many different leg and body motions including, but not limited to, walking, bending, standing and moving in bed. In various implementations, Y-shaped and X-shaped straps work through a loop, clevis, or pulley to self-adjust the strap support for the leg brace around the anchoring pivot point across various pelvic rotation motions and bending motions of the user.

This summary is not intended to identify key or essential features of the claimed subject matter, nor is it intended to be used as an aid in limiting the scope of the claimed subject matter.

### BRIEF DESCRIPTION OF THE DRAWINGS

FIG. 1 is a diagram of an example strap member for securing a leg brace or leg support with a clip fastening mechanism.

FIG. 2 is a diagram of an example strap member for securing a leg brace or leg support with a clasp fastening mechanism.

FIG. 3 is a diagram of an example strap member and belt member as a kit of single piece.

FIG. 4 is a diagram of an example belt member with adjustment buckle.

FIG. 5 is a diagram of an example strap member with clip, carabiner clip, and quick release buckle.

FIG. 6 is a diagram of an example strap member with multiple fastening mechanisms for attachment to a leg brace.

FIG. 7 is a diagram of an example leg brace with carabiner clip attachment held in reserve.

FIG. 8 is a diagram of a quick release buckle for attaching a strap member for securing a leg brace to a belt member.

FIG. 9 is a diagram of a no-slip clip of an example strap member.

FIG. 10 is a diagram of an example carabiner member for fastening a loop of an example leg brace.

FIG. 11 is a diagram of an example loop arrangement using a carabiner clip.

FIG. 12 is a diagram of an example securing clip or clasp to prevent slippage of an adjustable strap member.

FIG. 13 is a diagram of an example leg brace in relations to human leg anatomy.

FIG. 14 is a diagram of an example strap member configured for alignment with a human hip joint.

FIG. 15 is a diagram of an example strap system for securing an anchor loop of the strap system in alignment with a human hip joint.

FIG. 16 is a diagram of an example strap system including belt member for securing an anchor loop of the strap system in alignment with a human hip joint.

FIG. 17 is a diagram of an example strap system for securing a leg brace at multiple points.

FIG. 18 is a diagram of an example strap system for for securing an anchor loop of the strap system in alignment with a human hip joint, with straps for securing to a belt member at multiple points and to a leg brace at multiple points.

FIG. 19 is a diagram of an example strap system configured for aligning around a human hip joint and using a loop for attachment to the leg brace.

FIG. 20 is a diagram of an example strap system configured for aligning around a human hip joint with multiple attachment points on a leg brace.

FIG. 21 is a diagram of an example strap system with lateral strap for attaching to a belt member with an intervening tensioning spring or elastic member.

### DETAILED DESCRIPTION

In the following description, numerous details are set forth to provide an understanding of some embodiments of the present disclosure. It should be understood by those of ordinary skill in the art that the systems and methodologies may be practiced without these details and that numerous variations or modifications from the described embodiments may be possible.

This disclosure describes hold-up devices for securing a leg brace from sliding or rotating on a patient's leg. In one embodiment, an adjustable strap attaches to a belt that is around a patient's waist and connects in various ways to the leg brace to keep it from sliding down the patient's leg, from rotating on the patient's leg or from both sliding and rotating on the patient's leg.

In an implementation, the hold-up device has multiple attachment mechanisms on the distal end of the device to provide various ways of connecting to various types and models of leg braces.

For example, in one implementation, the hold-up device has one or more clips and one or more carabiner clips at its distal end for connecting to different parts of a leg brace or for connecting to different varieties of leg braces and appliances. Likewise, an example hold-up device may have multiple ways of attaching to the patient's belt, waistband or other belt-like member.

In various implementations, the hold-up device may take advantage of the anatomical location of the patient's hip joint to provide support for the leg brace which is anchored from common hinge points and hinge planes of the patient's own leg. This provides stability for the hold-up device since the length of the supporting strap does not need to vary in conjunction with a changing distance between a point on the waist and a point on the thigh caused by natural motions of the leg being braced. Instead, in some implementations the device can rely on a fixed distance from the patient's own hip joint to a point on the patient's leg, which remains constant. The example hold-up device can take advantage of this fixed distance, instead of basing support from waist or shoulder points that vary in distance from the leg brace as the patient moves.

In an implementation, a carabiner clip on the distal end of the hold-up device can connect to various clips at the top of a leg brace or onto a clip that is located further down the leg brace. Likewise, the carabiner clip can attach to other loops on the leg brace such as straps of hook-and-loop material built into a brace for securing the brace to the leg or can fasten to other clips and loops built onto the leg brace.

The distal end of the hold-up device may also include a clasp option that can be fasten directly to material at the top of the leg brace and hold the leg brace up against gravity.

When the hold-up device is a strap, the strap may be adjustable by having an adjustment buckle included along its length. In the same or another implementation, the hold-up device (which could be a strap) may include, or be made of, elastic material with or without the adjustment buckle, that keeps the lifting tension on the leg brace at a desired level.

In various embodiments, a strap system for securing a leg brace includes at least one strap member designed and configured to secure the leg brace to a belt member through a pivot point aligned on sagittal and transverse planes of the corresponding hip joint of the user. Locating an anchoring point of the securing leg brace in transecting planes of the actual hinging and pivoting motion of the corresponding human hip joint allows one or more securing straps to remain taut at a fixed length across many different leg and body motions including, but not limited to walking, bending, standing and moving in bed. In various implementations, Y-shaped and X-shaped straps work through a loop, clevis or pulley to self-adjust the strap support for the leg brace around the anchoring pivot point across various pelvic rotation motions and bending motions of the user.

The multiple attachment mechanisms may include one or more carabiner clips on the strap-like member for connecting to a loop or strap on the leg brace and a clip or a clasp on the strap-like member for grasping material of the leg brace. The hold-up device may have a buckle or an elastic material for adjusting the length or tension of the hold-up device. In an implementation, the hold-up device may have an auxiliary piece to base a lifting force of the hold-up device from the point of the hip joint point or the plane of the hip joint that is on the corresponding side that the patient is wearing the leg brace. In an implementation, locating the anchoring point for securing the leg brace in transecting planes of the actual hinging and pivoting motion of the corresponding hip joint allows one or more securing straps to remain taut at a fixed length with movement of the user including, but not limited to, walking, bending, standing and moving in bed. In various implementations, Y-shaped and X-shaped straps work through a loop, clevis or pulley to self-adjust support for the leg brace around the anchoring pivot point during various movements of the user.

FIG. 1 shows an example strap system including at least one strap member 100 and a belt member 102 providing an apparatus for securing a leg supporting member such as a leg brace 104 on a leg 50 of a user (patient) to prevent sliding and rotation of the leg brace 104 during its use. The leg brace 104 may be used when standing, bending, walking, moving in bed or during physical therapy or various activities including when recovering from an injury or surgery. The example strap member 100 may include a clip 106 at the distal end of the strap member 100. The clip can be attached to the material of the strap itself, or to a buckle or another strap 108 of the leg brace 104.

In FIG. 2, a clasp 200, such as a single loop clasp 200, double loop clasp, lobster clasp, swivel clasp, snap clip, reel clip clasp, etc., may be located at the distal end of the strap 100. The clasp 200 engages a ring, buckle, or hole of the leg brace 104. On the other hand, a clip 106, as in FIG. 1 grabs an edge of the material of the leg brace 104.

FIG. 3 shows an example strap member 100, belt member 102 and leg brace 104 removed from the leg 50 and waist of the user. In an implementation, the strap member 100 and belt member 102 may be used as a single system or kit, or as a single article that made as one piece.

FIG. 4 shows the belt member 102 with an adjustment buckle 400. The belt member 102 may include a piece that drops down to the area of the patient's hip joint to anchor support from a point or a plane associated with the hinging motion of the patient's hip joint, as shown in later Figures.

FIG. 5 shows one implementation of the strap member 100 with a belt clasp 500 at the proximal end and a carabiner clip 502 at the distal end. A mid-strap buckle 504 may be present to allow adjustment of the length or tension of the strap member 100.

In FIG. 6, the strap member 100, or a given strap system, may have various types of buckles on either end and may offer a choice of carabiner clips 502, clips 106, clasps 200 and other attachment mechanisms on the distal end of the strap member 100 to attach to a given leg brace 104 or to another strap or fastening mechanism of the leg brace 104.

The multiple attachment mechanisms provide a choice to the user for attaching to different places on a given leg brace 104, or for attaching to different types of leg braces 104.

FIG. 7 shows example strap member 100 attached to the belt member 102 by a clip 500 proximally and to the leg brace 104 by a clip 1-6 distally. In this version, the carabiner clip 502 is being held in reserve, and can be removed if the user does not want to use it.

FIG. 8 shows a close-up of an example quick release buckle 800 that attaches the strap member 100 to the belt member 102 proximally.

FIG. 9 shows an example no-slip clip 900 or clasp that attaches the strap 100 to the leg brace 104 distally.

FIG. 10 shows a same strap member 100 similar to that of FIGS. 8-9 with the strap 100 extended to attach to the leg brace 104 more distally with a carabiner clip 502 instead of with a clip 106 or clasp 200. The carabiner clip 502 can be connected to a clip, loop or strap of the leg brace 104 more distally (further down) closer to the patient's knee.

FIG. 11 shows the strap member 100 attached to the leg brace 104 distally with the carabiner clip 502 and loop arrangement.

FIG. 12 shows an example of a securing clip 1200 placed midway on the strap member 100 that is used to prevent slipping of the strap member 100 while the strap member 100 holds the leg brace 104 in place. The securing clip 1200 secures the adjustment buckle from slippage or undesirable self-adjusting, in one implementation.

FIG. 13 shows an example human leg 50 including a pivot point 1300 of the hip joint where the head of the femur (bone), which is located at the proximal end of the femur, hinges, rotates, and pivots in various directions in the hip socket; the acetabulum.

The location of the hip joint's natural pivot point 1300 (or pivot area) may be described as residing in multiple anatomical planes. For example, the pivot point 1300 resides in particular sagittal (front-to-back) longitudinal and transverse (horizontal) planes. Although the example strap systems described herein pivots at two of the planes: the sagittal and the transverse planes of pivot point 1300, the strap systems cannot pivot from all three planes in 3-dimensional space because the third plane, the coronal plane, would require a pivot point inside the human body. But by pivoting from the pivot point 1300 in two of the planes as they transect each other at the surface of the skin (or on the front of the clothes) at the front of the leg 50 or groin area, the example strap systems are able to achieve a securing system for the leg brace 104 that remains stable though many different motions of the leg 50. This stability results because the distance from the pivot point 1300 used by the example strap systems (outside the leg 50) to the top of the leg brace 104 remains constant. This is because the underlying pivoting femur (bone) provides a rigid, “linear” structure from approximately the same pivot point 1300 as the example strap system uses.

This anchoring scheme is different from conventional supports for leg braces which may attach at the shoulder or at the waist but do not avail to the pivot point 1300. Therefore the distance between the anchor point of any conventional support strap and the attachment spot on the leg brace changes as the leg or hip moves. However conventional support straps are of fixed length, so the leg brace 104 has to move slightly to compensate for the movement. This undesirable pull causing annoying rotation on the leg brace 104 that arises from conventional supports. In addition to the tendency of the leg brace to slip down the leg 50 when standing due to gravity.

FIG. 14 shows an example of a single strap system with a single strap 100 that proceeds over the pivot point between belt member 102 and leg brace 104. This mode keeps the leg brace 104 from sliding down the leg 50 and may provide enough support for some situations.

As an apparatus, such a strap system includes the leg support (brace) member 104 for immobilizing or bracing the knee or leg of the user, the belt member 102 that wraps around the user's waist, and at least one strap member 100 that secures the leg brace member 104 to the belt member 102 through the pivot point 1300 aligned on the sagittal and transverse planes of the corresponding hip joint of the user.

In an implementation, the leg support member 104, the belt member 102, and at least one strap member 100 may constitute a single article or appliance, or a “kit.”

In another implementation, the leg support member 104, the belt member 102 and at least one strap member 100 may be separate articles and the strap member 100 may have a first clasp 402 for grasping the leg support member 104 and a second clasp 404 for grasping the belt member 102 (or a conventional belt) at respective clasping locations configured to dispose the strap along the pivot point 1300.

FIG. 15 shows an implementation in which the strap is a 3-way “Y” strap 500, wherein a top part 502 of the 3-way Y strap 500 attaches to the belt member 102 at two points along the belt member 102 and is disposed through a loop, clevis or pulley 504 allowing the loop, clevis or pulley 504 to hang at the pivot point 1300 aligned on the sagittal and transverse planes of the corresponding hip joint of the user.

The loop, clevis or pulley 504 is free to move along the top part 502 (top strap) of the 3-way Y strap 500. A bottom part 506 of the 3-way Y strap 500 has a first end and a second end. The first end secures to the loop, clevis, or pulley 504 and the second end secures to the leg support member 104. The loop, clevis, or pulley 504 can slide along the top part 502 (top strap) during pelvic, waist or leg movements of the user, providing a self-compensating support of the leg brace 104 anchored at the pivot point 1300.

In an implementation, the top part 502 of the 3-way Y strap 500 is secured to the bottom part 506 of the 3-way Y strap 500 at the pivot point 1300, without the loop, clevis, or pulley 504. However, this implementation is not as forgiving of the user's movement.

FIG. 16 shows another implementation, similar to FIG. 17, in which the belt member 102 descends into a “V” shape 600 at the front centerline of the user's waist and one leg of the top part 502 of the 3-way Y strap 500 attaches to the bottom of the “V” shape 600 of the belt member 104. This configuration can add stability not only to the top part 502 of the 3-way Y strap 500, but can also keep the belt member 102 from rotating on the user's waist.

FIG. 17 shows another configuration in which the example strap is a 3-way strap 700, where a top part of the 3-way strap 700 comprises a single leg member 702. A first end of the leg member 702 is attachable to the belt member 102 at a location point along the belt member 102 and a second end of the leg member 702 is secured to a loop, clevis, or pulley 704 disposed at the pivot point 1300 that is aligned on the sagittal and transverse planes of the corresponding hip joint of the user.

The bottom part of 706 the example 3-way strap 700 is disposed through the loop, clevis, or pulley 704 and attaches to the leg support member 104 at two locations of the leg support member 104. The bottom part 706 of the 3-way strap 700 is free to slide with respect to the loop, clevis, or pulley 704 to compensate for movement of the user while stably maintaining the position of the loop, clevis, or pulley 704 at the pivot point 1300.

In a variation of FIG. 19, the top part 702 of the 3-way Y strap 700 is secured to the bottom part 706 of the 3-way Y strap 700 at the pivot point 1300 without the loop, clevis, or pulley 704. This example configuration is not as accommodating to the user's movement as when the loop, clevis or pulley 704 is present as a compensating element.

FIG. 18 shows an example implementation of the example strap support system and apparatus in which the strap system is a 4-way “X” strap 800. A top part 802 of the 4-way X strap 800 attaches to the belt member 102 at two locations on the belt member 102, and a bottom part 806 of the 4-way X strap 800 attaches to the leg support member 104 at two locations on the top aspect of the leg support member 104.

The top part 802 and the bottom part 806 of the 4-way X strap 800 meet at a single junction 804 at the pivot point 1300 that is aligned on sagittal and transverse planes of the corresponding hip joint of the user. One or more loop, clevis or pulley's 804 at the single junction 200 between the top part 802 and the bottom part 806 of the 4-way X strap 800 allows the top part 802 and the bottom part 806 to slide with respect to each other via one or more loops, clevises or pulleys 804. Since straps 802 and 806 of the 4-way “X” strap 800 can slide with respect to each other via one or more loops, clevises, or pulleys 804 this serves to keep the load bearing central junction 804 centered at the pivot spot 200 with movements of the user, thereby providing great support and stability to the leg brace 104.

In a variation, the top part 802 of the 4-way X strap 800 is secured to the bottom part 806 of the 4-way X strap 800 at the pivot point 1300 without a loop, clevis or pulley 804. This does not provide as much compensation for movement as with the one or more loops, clevises or pulleys 804 present but may be suitable in some circumstances.

FIG. 19 shows an implementation of the strap support system in which the support strap is a single strap 900 having a first end and a second end. The single strap disposed through a loop, clevis or pulley 902 that is attached to a top aspect of the leg support member 104. The first end and the second end of the single strap 900 are both attached to the belt member 102 at equal distances and/or equal angles with respect to the pivot point 1300 that is aligned on the sagittal and transverse planes of the corresponding hip joint of the user.

FIG. 20 is another variation for stability in which the strap system is a medial 1000 strap and a lateral strap 1002. A first end of the medial strap 1000 attaches to the belt member 102 at the medial aspect of the user's waist, and the second end of the medial strap 1000 attaches to the medial aspect of the leg support member 104. A first end of the lateral strap 1002 attaches to the belt member 102 at the lateral aspect of the user's waist and the second end of the lateral strap 1002 attached to the leg support member 104 at the lateral aspect of the leg support member 104. The medial strap 1000 and the lateral strap 1002 are disposed at opposing angles from each other.

FIG. 21 shows a variation of FIG. 20, in which the lateral strap 1002 attaches to the belt member 102 with an intervening tensioning spring or elastic member 1100. These compliant devices 1100 allow for some compensation of the length of the lateral strap 1002 due movement of the user.

In general, the example system is a belted strap for securing a leg brace including a belt member for wrapping around a waist of a user, at least one strap member for securing the leg brace to the belt member through a pivot point aligned on a sagittal plane and on a transverse plane of corresponding hip joint of the user.

The belted strap that has at least one strap member may be a single article or appliance. Or, the belted strap and at least one strap member may be separate articles. One or more strap members may have a first clasp for grasping the leg brace and a second clasp for grasping the belt member at respective clasping locations configured to dispose the strap along the pivot point.

The belted strap system may be a 3-way “Y” strap wherein a top part of the 3-way Y strap attaches to the belt member at two points along the belt member and is disposed through a loop, clevis or pulley allowing the loop, clevis or pulley to hang at the pivot point aligned on the sagittal and transverse planes of the corresponding hip joint of the user. Therefore the loop, clevis or pulley is free to move along the top part of the 3-way Y strap. The bottom part of the 3-way Y strap has a first end and a second end. The first end secures to the loop, clevis or pulley and the second end secures to the leg brace.

The belted strap may have a top part of the 3-way Y strap that is secured to the bottom part of the 3-way Y strap at the pivot point without the loop, clevis or pulley. The belted strap may also have a belt member that descends into a “V” shape at the front centerline of the user's waist and one leg of the top part of the 3-way Y strap attaches to the bottom of the “V” shape of the belt member for stability of both the straps and the belt member.

The top part of the 3-way Y strap may be a single leg member. The first end of the leg member is attachable to the belt member at one point along the belt member and the second end of the leg member is secured to a loop, clevis or pulley disposed at the pivot point aligned on the sagittal and transverse planes of the corresponding hip joint of the user. The bottom part of the 3-way Y strap can be disposed through the loop, clevis or pulley and attach to the leg brace at two locations. The bottom part of the 3-way Y strap is free to slide with respect to the loop, clevis or pulley to compensate for the user's movement while maintaining the position of the loop, clevis or pulley at the pivot point.

The top part of the 3-way Y strap can also be secured to the bottom part of the 3-way Y strap at the pivot point without the loop, clevis or pulley.

The belted strap assembly can also be a 4-way “X” strap where the top part of the 4-way X strap attaches to the belt at two places on the belt member and the bottom part of the 4-way X strap attached to the top of the leg brace at two places. The top part and the bottom part of the 4-way X strap meet at a single junction at the pivot point that is aligned on the sagittal and transverse planes of the corresponding hip joint of the user. One or more loops, clevises or pulleys at the single junction between the top part and the bottom part of the 4-way X strap allow the top and bottom parts to slide with respect to each other via one or more loops, clevises or pulleys.

The belted strap that is the 4-way X strap may have a top part that is secured to the bottom part at the pivot point without a loop, clevis, or pulley.

A version of the belted strap may have at least one strap that is a single strap having first and second ends. The single strap disposed through a loop, clevis or pulley that is attached to the top of the leg brace itself. The first end and the second end of the single strap are both attached to the belt member at equal distances and/or equal angles with respect to the pivot point and are aligned on the sagittal and transverse planes of the corresponding hip joint of the user.

An example belted strap may have a second end of a single strap that is attached to the belt member at a location more lateral with respect to the user than the attachment of the first end to the brace member. The second end of the single strap attaches to the belt member via an intervening tensioning spring or elastic member.

The belted strap assembly may be a medial strap and a lateral strap. The first end of the medial strap attaches to the belt member at the medial aspect of the user's waist and the second end of the medial strap attached to the medial aspect of a leg brace. The first end of the lateral strap attaches to the belt member at the lateral aspect of the user's waist and the second end of the lateral strap attached to the lateral aspect of the leg brace. The medial and lateral straps are disposed at opposing angles from each other.

The strap system for securing a leg brace may just be the straps themselves or just a single strap member for securing the leg brace to a belt member through a pivot point aligned on sagittal and transverse planes of the corresponding hip joint of a user. Thus, the strap member and the belt member may be separate articles, the strap member also including a first clasp for grasping the leg brace and a second clasp for grasping the belt at respective clasping locations configured to dispose the strap along the pivot point.

The strap system that is just the strap can also be a 3-way “Y” strap where the top part of the 3-way Y strap attaches to the belt member at two points along the belt member and is disposed through a loop, clevis or pulley allowing the loop, clevis or pulley to hang at the pivot point aligned on sagittal and transverse planes of the corresponding hip joint of the user. The loop, clevis or pulley is free to move along the top part of the 3-way Y strap. The bottom part of the 3-way Y strap has first and second ends. The first end secures to the loop, clevis or pulley and the second end secures to the leg brace. The top part of the 3-way Y strap can be secured to the bottom part of the 3-way Y strap at the pivot point without the loop, clevis or pulley. The belt member can descends into a “V” shape at the front centerline of the user's waist and one leg of the top part of the 3-way Y strap attaches to the bottom of the “V” shape of the belt member.

The 3-way Y strap may have a single leg member. The first end of the leg member is attachable along the belt member and the second end of the leg member is secured to a loop, clevis or pulley disposed at the pivot point aligned on the sagittal and transverse planes of the corresponding hip joint of the user. The bottom part of the 3-way Y strap is disposed through the loop, clevis or pulley and attaches to the leg brace at two locations. The bottom part of the 3-way Y strap is free to slide with respect to the loop, clevis or pulley to compensate for movement of the user while maintaining the position of the loop, clevis or pulley at the pivot point. The top part of the 3-way Y strap may be secured to the bottom part of the 3-way Y strap at the pivot point without the loop, clevis or pulley.

The strap may be a 4-way “X” strap where the top part of the 4-way X strap attaches to the belt at two places and the bottom part of the 4-way strap attached to the top of the leg brace at two places. The top and bottom parts of the 4-way X strap meet at a single junction at the pivot point aligned on the sagittal and transverse planes of the corresponding hip joint of the user. One or more loops, clevises or pulleys at the single junction between the top part and the bottom part of the 4-way X strap allow the top and bottom parts to slide with respect to each other via one or more loops, clevises or pulleys. The top part of the 4-way X strap can also be secured to the bottom part of the 4-way X strap at the pivot point without a loop, clevis or pulley.

As a single strap, the system can have a first end and a second end. The single strap is disposed through a loop, clevis or pulley that is attached to the top of the leg brace. The first and second ends of the single strap are both attached to the belt member at equal distances and/or equal angles with respect to the pivot point that is aligned on the sagittal and transverse planes of the corresponding hip joint of the user. The second end of the single strap attaches to the belt member at a location on the belt member more lateral with respect to the user than the attachment of the first end to the belt member. The second end of the single strap can attach to the belt member via an intervening tensioning spring or elastic member.

As straps, the system can be a medial strap and a lateral strap. The first end of the medial strap attaches to the belt member at the medial aspect of the user's waist and the second end of the medial strap attached to the medial aspect of the leg brace. The first end of the lateral strap attaches to the belt member at a lateral aspect of the user's waist and the second end of the lateral strap attached to the lateral aspect of the leg brace. The medial strap and the lateral strap are disposed at opposing angles from each other.

### CONCLUSION

Although a few embodiments of the disclosure have been described in detail above, those of ordinary skill in the art will readily appreciate that many modifications are possible without materially departing from the teachings of this disclosure. Accordingly, such modifications are intended to be included within the scope of this disclosure as defined in the claims.

1. A hold-up device for securing a leg brace, comprising:
an adjustable strap, straps or strap-like member attachable to a belt, waistband or belt-like member around a patient's waist, the hold-up device connectable in various ways to the leg brace to keep the brace from sliding down or rotating on the patient's leg or from both sliding and rotating on the patient's leg.

2. The hold-up device of claim 1 wherein the strap-like member has multiple attachment mechanisms on the distal end of the strap-like member to provide various ways of connecting to different locations on a given leg brace or for connecting to various types or models of a leg brace.

3. The hold-up device of claim 2 wherein the multiple attachment mechanisms include a choice of one or more carabiner clips on the strap-like member for connecting to a loop or strap on the leg brace and a clip or a clasp on the strap-like member for grasping material of the given leg brace.

4. The hold-up device of claim 1, further comprising a buckle or an elastic material for adjusting the length or tension of the hold-up device.

5. The hold-up device of claim 1, where an auxiliary piece of the hold-up device is configured to base a lifting force of the hold-up device in alignment with a hip joint or a hip joint plane.

6. An apparatus comprising: a leg support member for immobilizing the user's knee or leg or for bracing the user's knee or leg;
a belt member for wrapping around the waist of the user; and
at least one strap member configured to secure the leg support member to the belt member through a pivot point aligned on sagittal and transverse planes corresponding to the user's hip joint.

7. The apparatus of claim 6 where the leg support member, the belt member and at least one strap member comprise a single article or appliance.

8. The apparatus of claim 6 where the leg support member, the belt member and at least one strap member comprise separate articles, at least one strap member comprising a first clasp for grasping the leg support member and a second clasp for grasping the belt member at respective clasping locations configured to dispose the strap along the pivot point.

9. The apparatus of claim 6, where at least one strap comprises a 3-way “Y” strap where the top part of the 3-way Y strap attaches to the belt member at two points and is disposed through a loop, clevis or pulley allowing the loop, clevis or pulley to hang at the pivot point that is aligned on the sagittal and transverse planes of the user's corresponding hip joint;
wherein the loop, clevis or pulley is free to move along the top part of the 3-way Y strap; and
wherein a bottom part of the 3-way Y strap has first and second ends. The first end secures to the loop, clevis or pulley and the second end secures to the leg support member.

10. The apparatus of claim 9, wherein the top part of the 3-way Y strap is secured to the bottom part of the 3-way Y strap at the pivot point without the loop, clevis or pulley.

11. The apparatus of claim 10, wherein the belt member descends into a “V” shape at the front centerline of the user's waist and one leg of the top part of the 3-way Y strap attaches to the bottom of the “V” shape of the belt member.

12. The apparatus of claim 6, wherein at least one strap comprises a 3-way strap. A top part of the 3-way Y strap comprises a single leg member. The first end of the leg member is attachable at one point along the belt member and a second end of the leg member is secured to a loop, clevis or pulley disposed at the pivot point aligned on the sagittal and transverse planes of the corresponding hip joint of the user;
wherein a bottom part of the 3-way Y strap is disposed through the loop, clevis or pulley and attaches to the leg support member at two locations of the leg support member; and
wherein the bottom part of the 3-way Y strap is free to slide with respect to the loop, clevis or pulley to compensate for movement of the user while maintaining the position of the loop, clevis or pulley at the pivot point.

13. The apparatus of claim 12, wherein the top part of the 3-way Y strap is secured to the bottom part of the 3-way Y strap at the pivot point without the loop, clevis or pulley.

14. The apparatus of claim 6, wherein at least one strap comprises a 4-way “X” strap. The top part of the 4-way X strap attaches at two places on the belt member and the bottom part of the 4-way X strap attached to the top of the leg support member at two places;
wherein the top part and the bottom part of the 4-way X strap meet at a single junction at the pivot point aligned on the sagittal and transverse planes of the corresponding hip joint of the user; and
wherein one or more loops, clevises or pulleys at the single junction between the top and bottom parts of the 4-way X strap allow the top and bottom parts to slide with respect to each other via one or more loops, clevises or pulleys.

15. The apparatus of claim 14, wherein the top part of the 4-way X strap is secured to the bottom part of the 4-way X strap at the pivot point without a loop, clevis or pulley.

16. The apparatus of claim 6, where at least one strap comprises a single strap having first and second ends;
the single strap disposed through a loop, clevis or pulley attached to the top aspect of the leg support member; and
wherein the first and second ends of the single strap are both attached to the belt member at equal distances and/or equal angles with respect to the pivot point aligned on the sagittal and on the transverse planes of the corresponding hip joint of the user.

17. The apparatus of claim 16, wherein the second end of the single strap attaches at a location on the belt member more lateral with respect to the user than the attachment of the first end to the belt member; and
wherein the second end of the single strap attaches to the belt member via an intervening tensioning spring or elastic member.

18. The apparatus of claim 6, wherein at least one strap comprises a medial strap and a lateral strap;
wherein the first end of the medial strap attaches to the belt member at the medial aspect of the user's waist and the second end of the medial strap attached to the leg support member at the medial aspect of the leg support member;
wherein the first end of the lateral strap attaches to the belt member at the lateral aspect of the user's waist and the second end of the lateral strap attached to the lateral aspect of the leg support member; and
wherein the medial strap and the lateral strap are disposed at opposing angles from each other.

19. A belted strap for securing a leg brace, comprising:
a belt member for wrapping around the waist of a user;
at least one strap member for securing the leg brace to the belt member through a pivot point aligned on sagittal and transverse planes of the corresponding hip joint of the user.

20. The belted strap of claim 19, wherein the belt member and the at least one strap member comprise a single article or appliance.
